(19) United States
(12) Patent Application Publication
(10) Pub. No.: US 2021/0395809 A1
(43) Pub. Date: Dec. 23, 2021

(54) METHOD FOR DETECTING AN AMPLIFICATION PHASE IN AN AMPLIFICATION

(71) Applicant: Analytik Jena GmbH, Jena (DE)

(72) Inventor: Eva Möller, Jena (DE)

(21) Appl. No.: 17/348,846

(22) Filed: Jun. 16, 2021

(30) Foreign Application Priority Data
Jun. 18, 2020 (DE) 10 2020 116 178.6

(51) Int. Cl. C12Q 1/6851; G16B 25/00; G06F 17/18

(52) U.S. Cl. CPC C12Q 1/6851; G06F 17/18; G16B 25/00

(57) ABSTRACT

A method for detecting whether an amplification phase has occurred in an amplification in which a nucleic acid sequence is increased in a defined number of amplification cycles includes determining the intensity of a signal of the nucleic acid sequence after each amplification cycle and representing the determined intensity as a measurement point as a function of the amplification cycle. The method includes calculating a first regression line from a first measurement point recorded after a first amplification cycle to the determined starting point of the amplification phase; calculating a second regression line over all measurement points; comparing the first regression line to the second regression line regarding the dissimilarity of the first and second regression lines using a first statistical test, wherein a p-value is ascertained; and detecting whether an amplification phase has occurred based on whether the p-value is below or above a defined limit value.

### CROSS-REFERENCE TO RELATED APPLICATIONS

The present application is related to and claims the priority benefit of German Patent Application No. 10 2020 116 178.6, filed on Jun. 18, 2020, the entire contents of which are incorporated herein by reference.

### TECHNICAL FIELD

The present disclosure relates to a method for detecting whether an amplification phase has taken place in an amplification in which at least one nucleic acid sequence is increased in a defined number of amplification cycles, the intensity of a signal of the at least one nucleic acid sequence being ascertained after each amplification cycle and the ascertained intensity being represented as a measurement point as a function of the amplification cycle.

### BACKGROUND

An amplification is an increase of at least one nucleic acid sequence, for example, a DNA sequence. Amplification of nucleic acid sequences can be a naturally occurring process within genetics. In addition, nucleic acid sequences are deliberately increased in vitro in molecular biology in order to, for example, clone genes or detect diseases. Aside from various isothermal methods for amplification, the polymerase chain reaction (PCR) may be considered the most vital method of amplification.

In a PCR, a nucleic acid sequence, generally a short section of the DNA, is increased by means of an enzyme in a defined number of amplification cycles. The enzyme used is typically a so-called DNA polymerase. After each amplification cycle, the nucleic acid sequence is present in double the number so that the number of nucleic acid sequences increases exponentially with the number of amplification cycles. A typical number of amplification cycles is about 20-40 amplification cycles.

If, in addition to the pure increase of the nucleic acid sequence, a quantitative analysis of the amplification is also desired, one of the numerous variations of PCR can be used, such as real-time PCR, which is also referred to as quantitative PCR (qPCR). Here, before the start of amplification, a marker is added to the nucleic acid sequences and specifically binds to the nucleic acid sequences and, bound there, emits fluorescence. In this way, the intensity of the fluorescence of the markers bound to the nucleic acid sequences can be determined in real time after each amplification cycle. In the ideal case, the determined intensity of the fluorescence, which is plotted over the amplification cycles, has a sigmoidal behavior. In the first amplification cycles, initially only one background signal is measured until the nucleic acid sequences are sufficiently increased. With the exponentially increasing number of nucleic acid sequences, the intensity of the fluorescence increases sharply before it enters a plateau in which no further increase of the nucleic acid sequences takes place. Since the measured intensity of the fluorescence of the bound markers is thus proportional to the number of nucleic acid sequences, different models can be used for quantifying the amplification.

The so-called threshold cycle (Ct) indicates, for example, that amplification cycle in which the intensity of the fluorescence of the markers for the first time increases significantly above the (fluorescence) background signal. A low Ct value thus means that a large number of nucleic acid sequences are present at an early point in time. Typically, no single amplification is performed, but rather a whole series of amplifications is performed in parallel and preferably evaluated in parallel. For the determination of the Ct value, this means that for all amplifications performed, a common threshold value must be found, based on which the Ct value is determined.

For the determination of the so-called quantification cycle (Cq), a sigmoidal model function is adapted to the measurement points determined during the amplification and is used as the basis for ascertaining the phase of the exponential increase in the fluorescence. The Cq value can be determined as an alternative to the Ct value. Although the approach of determining the two values is different, the same amplification cycle should be obtained in the ideal case as the Cq value and the Ct value. If a series of amplifications is performed, the Cq value and the Ct value should ideally match for a respective amplification, different values of the Cq value and Ct value frequently being obtained within the series of amplifications for different amplifications.

The determination of the threshold cycle (Ct) and of the quantification cycle (Cq) proves to be particularly difficult if the amplification phase is only weakly pronounced and therefore difficult to identify. Particularly in this case, deviations of the quantification cycle (Cq) from the threshold cycle (Ct) frequently occur.

U.S. Ser. No. 10/176,293 B2 specifies a method for systematically determining a quantification parameter from an amplification.

Patent specification EP 1472518 B1 describes a method for evaluating a qPCR, wherein a plurality of amplifications is to be evaluated in parallel. In this case, a region of an exponential intensity increase with a lower and an upper limit as well as a background line are determined. In addition, a limit value, which lies between the lower and the upper limit of the intensity increase, is determined across all amplifications. This limit value indicates an amplification cycle which is referred to as a threshold cycle and which serves to quantitatively compare the individual amplifications with one another.

CA 02736243 A1 describes a further method for evaluating a PCR. In one step, it is checked whether an amplification phase is present. For example, series of measurements that are too short or artifacts which only seem to represent an amplification are sorted out.

EP 1 647 910 A1 discloses that the intensity of the fluorescence is described with a regression function which contains a linear and a non-linear function, in particular a sigmoid function. The coefficient of the sigmoid function is subsequently subjected to a t test in order to check with which probability it is not equal to zero, i.e., how likely it is that the intensity of fluorescence has a non-linear increase. Measurements without an amplification phase are only sorted out afterwards. The regression function also contains four parameters, as a result of which the regression gains uncertainty.

### SUMMARY

The object to be achieved by the present disclosure is to provide a method which detects in a simple manner whether an amplification phase has taken place in an amplification.

The object is achieved according to the present disclosure by a method for detecting whether an amplification phase has taken place in an amplification in which at least one nucleic acid sequence is increased in a defined number of amplification cycles, the intensity of a signal of the at least one nucleic acid sequence being ascertained after each amplification cycle and the ascertained intensity being represented as a measurement point as a function of the amplification cycle, wherein the method comprises the following steps:
Determining a starting point of the amplification phase based on the ascertained measurement points, the starting point marking the transition between a substantially constant background signal and a beginning of the amplification phase,
Calculating a first regression line from a first measurement point recorded after a first amplification cycle to the determined starting point of the amplification phase,
Calculating a second regression line over all measurement points,
Comparing the first regression line to the second regression line with regard to the dissimilarity of the first regression line and the second regression line by means of a first statistical test, wherein a first application-specific p-value is ascertained,
In the event that the ascertained first application-specific p-value is above a defined limit value:
   Detecting that no amplification phase has taken place,
   Aborting a further evaluation,
In the event that the ascertained first application-specific p-value is below a defined limit value:
   Detecting that an amplification phase has taken place,
   Performing a further evaluation.

The method according to the present disclosure firstly includes determining the starting point of the amplification phase, wherein the determined starting point may possibly be different depending on the method used. It goes without saying that the person skilled in the art does not consider artifacts and other measurement points that are obviously faulty within the scope of the method according to the present disclosure. Subsequently, the first and the second regression line are calculated. This is done in a known manner by a linear regression based on the respective measurement points, wherein a maximum of the respective adaptation quality of the first and the second regression line is to be achieved. The first regression line is calculated between the first measurement point and the determined starting point of the amplification phase so that the first regression line describes a background intensity of the fluorescence. The second regression line is calculated over all measurement points and thus includes an amplification phase if an amplification phase has taken place.

In the next step, the first and the second regression line are compared with regard to their dissimilarity by means of the first statistical test, for example based on the respective slope of the first and the second regression line. In particular, the measurement points between the first measurement point and the determined starting point of the amplification phase are used for this purpose. In the event that an amplification phase has taken place, the first and the second regression line should differ from one another since the amplification phase has a non-linear increase in contrast to the fluorescence background.

As a rule, it is rather expected that an amplification phase took place in an amplification. In statistics, the null hypothesis is defined as the result that is opposite to the expected result, that is, in this case, that no amplification phase has taken place. Applied to the comparison of the first and the second regression line, one would rather expect the two to differ from one another. The corresponding null hypothesis of the first statistical test is thus that the first and the second regression line are similar.

The first statistical test outputs a first application-specific p-value, based on which it is detected relative to a defined limit value whether an amplification phase has taken place. A lower first application-specific p-value means that the first and the second regression line are sufficiently different so that an amplification phase can be inferred. An increased first application-specific p-value signals that the first and the second regression line are similar to one another and that no amplification phase has thus taken place.

The method according to the present disclosure thus makes it possible in a simple manner to detect whether or not an amplification phase has taken place. If it is detected that no amplification phase has taken place, the further evaluation is aborted and no further time and energy is put into the evaluation. If, on the other hand, it is detected that an amplification phase has taken place, a further evaluation of the measurement points can take place in order to obtain quantitative and/or qualitative information about the amplification phase. Within diagnostic applications, it can typically be expected that 60-80% of the evaluated amplifications contain an amplification phase. Thus, by means of the method according to the present disclosure, it can be detected in advance for up to 40% of the amplifications performed that no amplification phase has taken place, and the further evaluation can be saved.

In addition, the method according to the present disclosure offers the advantage that the person skilled in the art attains a certainty that the further evaluation is useful for a detected amplification phase, whereby the trustworthiness of the subsequent results of the further evaluation increases. This applies in particular to those amplifications in which the amplification phase is difficult to identify since the measurement points fluctuate greatly, for example.

The limit value can, for example, be oriented towards usual limit values for p-values. Typically, a series of amplifications is performed in parallel. In addition, positive and negative controls which serve to check a systematic error in the measured series of amplifications are measured within this series. For a positive control, a small first application-specific p-value is expected, whereas for a negative control, a high first application-specific p-value is expected. Thus, if the first application-specific p-value of a positive control exceeds the limit value, all further amplifications of the same series can be considered to have errors and are not further evaluated. Conversely, the series of amplifications is also considered to be faulty if a first application-specific p-value below the limit value is determined for a negative control within the series.

Alternatively, the limit value can be ascertained based on the first application-specific p-values of at least one positive control and/or at least one negative control within a series of amplifications. It is detected in this way that an amplification phase has taken place when a first application-specific p-value of an amplification is less than the lowest first application-specific p-value within the first application-specific p-values determined for the at least one negative control, or when a first application-specific p-value is less than the highest first application-specific p-value within those first application-specific p-values determined for the at least one positive control.

In one possible development, the method comprises the following steps:
Adapting a first model function, which contains at least one sigmoid function, to all measurement points in a first step,
Adapting a quadratic model function to all measurement points in a second step,
Comparing the first model function to the quadratic model function with regard to the dissimilarity of the first model function and the quadratic model function by means of a second statistical test, wherein a second application-specific p-value is ascertained,
In the event that the ascertained second application-specific p-value is above a defined limit value:
   Detecting that no amplification phase has taken place,
In the event that the ascertained second application-specific p-value is above a defined limit value:
   Detecting that an amplification phase has taken place.

In the further evaluation, e.g., in the case of a detected amplification phase, it is examined whether the expected sigmoidal progression of the amplification phase is actually present. For this purpose, the first model function, which is to represent the expected sigmoidal progression of the amplification phase, is compared to the quadratic model function, which has no continuous exponential increase. The expectation is, therefore, that a difference between the quadratic and the first model function is found. The null hypothesis is, therefore, that the quadratic model function and the first model function are similar. This null hypothesis is subsequently checked by means of a second statistical test, wherein a second application-specific p-value is obtained. Depending on the position of the second application-specific p-value relative to a defined limit value, it is detected that an amplification phase has occurred or not.

While the first statistical test checks whether a non-linear increase is included in the amplification and thereby estimates whether an amplification phase has taken place, the second statistical test checks the exponential increase of the amplification phase. By means of the first application-specific p-value, a preselection of those amplifications in which an amplification phase is likely to have taken place can thus be made. The second application-specific p-value provides a statement about the presence of an exponential increase in the amplification. Thus, the first and the second application-specific p-value are obtained and both can be used to establish an amplification phase.

In a development of the method according to the present disclosure, the first regression line and the second regression line are compared with regard to the dissimilarity of the first regression line and second regression line based on the variances of the residuals of the measurement points to the first regression line and to the second regression line by means of the first statistical test. Thus, firstly, the residuals of the measurement points to the first and to the second regression line are formed, and subsequently the variances of the residuals of the measurement points. The first statistical test analyzes the variances of the residuals of the measurement points to the first and to the second regression line for differences.

Advantageously, an F-test is used as the first statistical test. An F-test serves to generally check differences between two statistical populations based on the variances of the two statistical populations. For example, the F-test checks the variances of the residuals of the measurement points to the first regression line and to the second regression line for differences.

In another development of the method according to the present disclosure, the first model function and the quadratic model function are compared with regard to the dissimilarity of the first model function and the quadratic model function based on the variances of the residuals of the measurement points to the first model function and to the quadratic function by means of the second statistical test. The second statistical test analyzes, analogously to the first statistical test, the variances of the residuals of the measurement points to the first and to the quadratic model function for differences.

Preferably, an F-test is used as the second statistical test. For example, the F-test checks the variances of the residuals of the measurement points to the first model function and to the quadratic model function for differences.

A possible development provides that the first model function is formed as a sum of a sigmoid function and a linear function. In this case, the sigmoid function represents the progression of the amplification phase, while the linear function describes a background intensity. Since the first regression line represents the background intensity, the first regression line can preferably be used as a linear function for the first model function.

In another development, a p-value of 0.05 or of 0.01 or between 0.01 and 0.05 is preferably defined as the limit value of the ascertained first and/or second application-specific p-value. The limit value is typically oriented towards the generally customary thresholds or threshold values for p-values.

In another possible development, the first regression line is subtracted as background line from the entirety of the measurement points. After the second statistical test and upon confirmation of the presence of an amplification phase, the first regression line is typically subtracted as background line from all measurement points, thus eliminating the background intensity. This subsequently facilitates the determination of a quantification parameter.

The fluorescence or absorbance of the at least one nucleic acid sequence is advantageously measured as the signal intensity of the at least one nucleic acid sequence. In a relevant wavelength range, the at least one nucleic acid sequence frequently has no or hardly any absorbance which could be used for the evaluation of an amplification. The nucleic acid sequences are, therefore, frequently combined with marker molecules that incorporate into the nucleic acid sequences and have a fluorescence.

A polymerase chain reaction is preferably used as the amplification technique. In particular, real-time PCR or qPCR is used.

In a preferred development, the value of the ascertained first and/or second application-specific p-value is used in order to determine the quality of at least one quantification parameter, wherein the at least one quantification parameter is a measure for the concentration of the nucleic acid sequences and is determined based on the intensity of the signal of the nucleic acid sequences. The first and/or the second application-specific p-value is not to replace the at least one quantification parameter but offers a measure for the reliability and trustworthiness of the determined quantification parameter. For example, a quantification parameter associated with a low first application-specific p-value may be considered to be more reliable than a quantification parameter associated with a comparatively high first application-specific p-value.

Advantageously, a threshold cycle (Ct value) and/or a quantification cycle (Cq value) is determined as at least one quantification parameter. In particular, if the Ct value deviates from the Cq value of the same amplification, it can be checked by means of the first and/or the second application-specific p-value how reliably an amplification phase was detected and/or how well the sigmoidal progression of the amplification phase could be adapted. When comparing different amplifications to different Ct values or Cq values, the first and/or the second application-specific p-value can also be used to establish a cause of the different Ct values or Cq values. For example, Cq values in which a very low first and second application-specific p-value has been determined can be considered more reliable than those Cq values in which the first and the second application-specific p-value are comparatively higher.

A possible development of the method according to the present disclosure provides that the starting point of the amplification is determined by means of a method of a sliding window, wherein a defined number of measurement points ascertained in succession is taken from all measurement points and defined as a window, wherein a specific parameter is determined in each of the first and second halves of the window, wherein the window is then shifted by a measurement point and a specific parameter is again determined in each of the first and second halves of the window, wherein the window is shifted by a measurement point in each case after the determination of the specific parameters until all possible windows have been considered and all respective specific parameters have been determined, wherein the measurement point at which the difference between the specific parameters from the first and second halves of the window is greatest is selected as the starting point.

In a possible development, an average value of the measurement points or a maximum value of the measurement points or an increase of a line which is formed between a first measurement point of the window and a middle measurement point of the window and between the middle measurement point of the window and a last measurement point of the window is calculated as a specific parameter.

### BRIEF DESCRIPTION OF THE DRAWINGS

The method according to the present disclosure is described below with reference to cited FIGS. 1-5. The following are shown:

FIG. 1 shows an exemplary embodiment of the method according to the present disclosure;

FIGS. 2a and 2b shows in each case a graph of an amplification in which an amplification phase is detected;

FIGS. 3a and 3b shows in each case a graph of an amplification in which no amplification phase is detected;

FIGS. 4a and 4b shows in each case a graph of an amplification in which an amplification phase is not clearly detected; and

FIG. 5 shows a graph of an amplification in which the method of sliding windows is used in a determined manner for determining the starting point of the amplification phase.

### DETAILED DESCRIPTION

The method according to the present disclosure can be used for all amplifications, irrespective of the technique used or the apparatus used. The method according to the present disclosure serves to detect whether an amplification phase has taken place in an amplification in which at least one nucleic acid sequence is increased in a defined number of amplification cycles. After each amplification cycle, the intensity of a signal of the at least one nucleic acid sequence is ascertained and the ascertained intensity is represented as a measurement point as a function of the amplification cycle. For example, the fluorescence or absorbance of the at least one nucleic acid sequence can be measured as a signal whose intensity is ascertained. The amplification can be performed, for example, with the polymerase chain reaction technique.

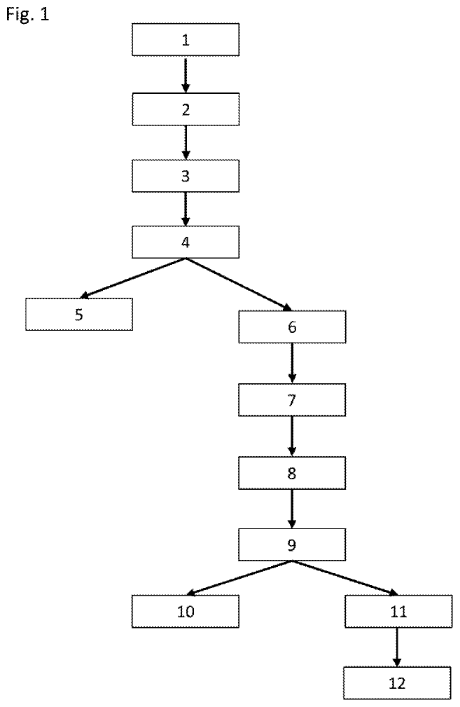

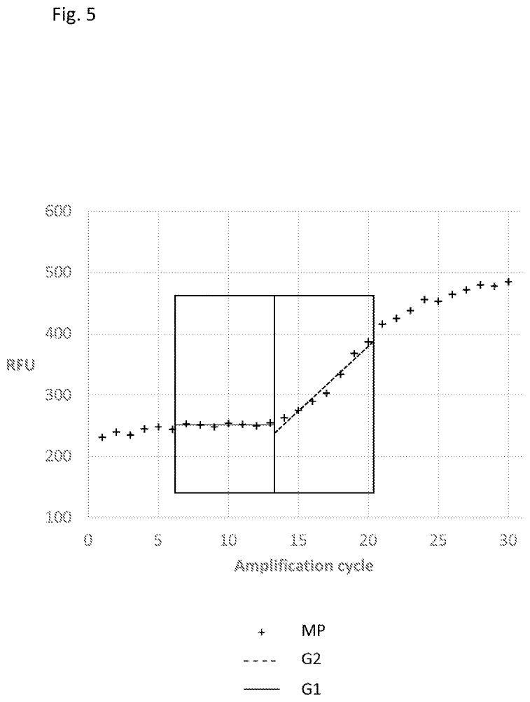

FIG. 1 shows an exemplary embodiment of the method according to the present disclosure. In the first step 1, a starting point of the amplification phase is determined based on the ascertained measurement points MP, the starting point marking the transition between a substantially constant background signal and a beginning of the amplification phase. A possible method for determining the starting point of the amplification phase is explained in more detail in FIG. 5. A first regression line R1 is then calculated in a second step 2 from a first measurement point MP, which was recorded after a first amplification cycle, to the determined starting point SP of the amplification phase. In the third step 3, a second regression line R2 is calculated over all measurement points MP.

The first regression line R1 is compared to the second regression line R2 with regard to the dissimilarity of the first regression line R1 and the second regression line R2 by means of a first statistical test (step 4). In particular, the measurement points MP between the first measurement point and the starting point SP of the amplification phase are used. For example, by means of the first statistical test, the first regression line R1 and the second regression line R2 can be compared with regard to the dissimilarity of the first regression line R1 and the second regression line R2 based on the variances of the residuals of the measurement points MP to the first regression line and to the second regression line. From the first statistical test, for example an F-test, a first application-specific p-value is ascertained. If the ascertained first application-specific p-value is above a defined limit value, it is detected in step 5 that no amplification phase has taken place and a further evaluation is aborted.

However, if the ascertained first application-specific p-value is below a defined limit value, it is detected in step 6 that an amplification phase has taken place and the evaluation of the amplification is continued. In a seventh step 7, a first model function F1 is then adapted to all measurement points MP. The first model function F1 contains a sigmoid function and optionally additionally a linear function. The first regression line R1 in particular can be used as a linear function. In step 8, a quadratic model function F2 is adapted to all measurement points MP.

In step 9, similarly to step 4, the first model function F1 and the quadratic model function F2 are compared with regard to their dissimilarity by means of a second quadratic test, wherein a second application-specific p-value is ascertained. Optionally, the comparison of the first model function F1 to the quadratic model function F2 can be compared with regard to their dissimilarity based on the variances of the residuals of the measurement points MP to the first model function and to the quadratic function by means of the second statistical test. The second statistical test may, for example, be an F-test.

If the ascertained second application-specific p-value is above a defined limit value, it is detected in step 10 that no amplification phase has taken place. However, if the ascertained second application-specific p-value is below a defined limit value, it is detected in step 11 that an amplification phase has taken place. The limit value of the ascertained first and/or second application-specific p-value is, for example, defined as a p-value of 0.05 or of 0.01 or between 0.01 and 0.05. The limit value can also be established in a series of amplifications using at least one positive control and/or negative control. In step 12, for example, the regression line R1 is subtracted as background line from the amplification curve for the subsequent optional determination of a quantification parameter.

The ascertained first and/or second application-specific p-value can optionally be used to determine the quality of at least one quantification parameter, wherein the at least one quantification parameter is a measure for the concentration of the nucleic acid sequences and is determined based on the intensity of the signal of the nucleic acid sequences. The at least one quantification parameter may be, for example, a threshold cycle (Ct value) and/or a quantification cycle (Cq value).

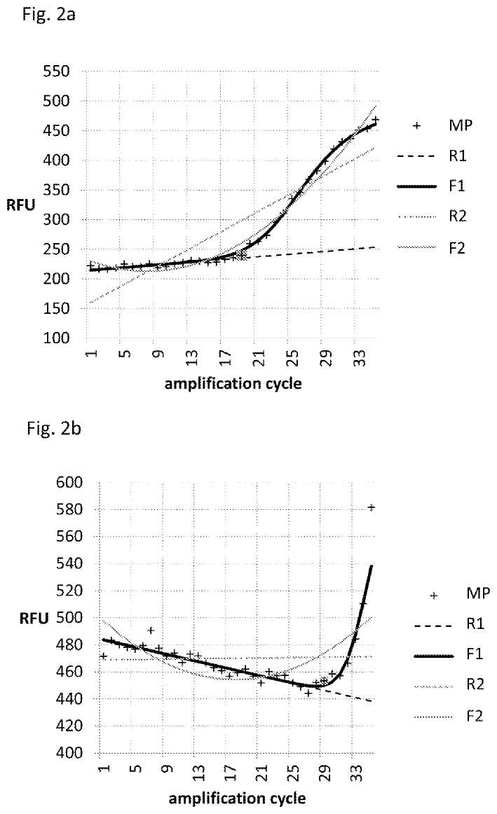

FIGS. 2a-4b show exemplary amplifications evaluated using the method according to the present disclosure. The determined (relative) intensity of the fluorescence of a nucleic acid sequence (RFU) was plotted as measurement points MP over the amplification cycle. The following can be seen in each case: the first regression line R1, the second regression line R2, the first model function F1, and the quadratic model function F2. The ascertained first and second application-specific p-values p1, 2 are compiled in Table 1. The starting point of the amplification phase has a gray background in each case.

In FIGS. 2a and 2b, a value of less than 0.01 was obtained as first application-specific p-value p1 for the respective amplification. This very low first application-specific p-value p1 indicates that an amplification phase has taken place. Therefore, a further evaluation was performed and the measurement points MP were adapted with the first model function F1 and the quadratic model function F2. Obtained from the second statistical test was a second application-specific p-value p2, which is less than 0.01 for each of the two amplifications in FIGS. 2a and 2b. Due to the very low first and second application-specific p-values p1, p2, it can be inferred with high certainty in FIGS. 2a and 2b that an amplification phase has taken place. If a quantification parameter were now determined based on FIGS. 2a and 2b, the determined value of the quantification parameter would be considered to be very trustworthy.

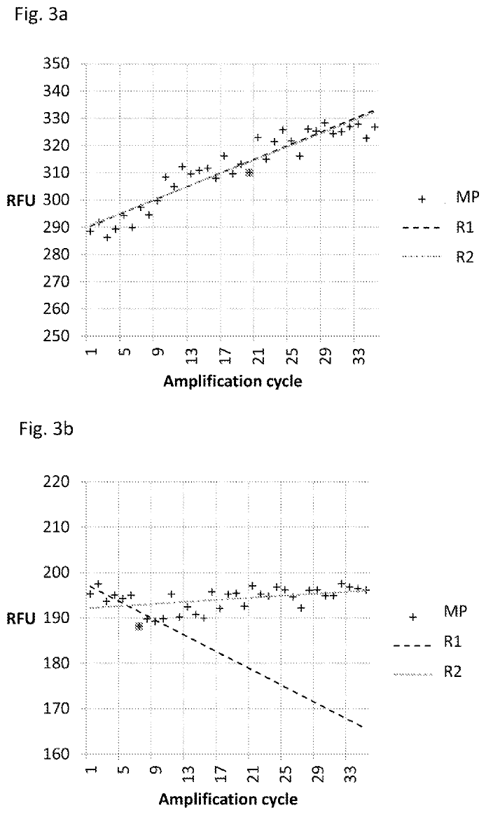

FIGS. 3a and 3b each show an amplification in which no amplification phase has taken place. Performing the first statistical test based on the first and second regression lines R1, R2 resulted in a first application-specific p-value p1 of 0.99 in FIG. 3a and in a first application-specific p-value p1 of 0.1 in FIG. 3b. These two values for the first application-specific p-value p1 correspond to rather high values, which is why it has been postulated that no amplification phase has taken place and no further evaluation was performed. If a quantification parameter was determined in these two examples, a high uncertainty would be ascribed to it or it can be considered to be false as a result of the high first application-specific p-value p1. In particular for automated amplification apparatuses in which a high number of amplifications is performed simultaneously, a high first application-specific p-value p1 can signal that a further evaluation is no longer possible, thus significantly reducing the time expenditure of the further evaluation.

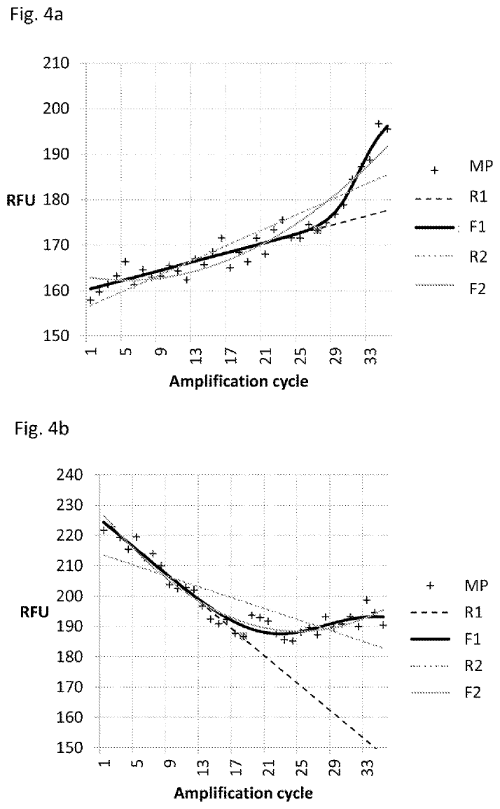

However, it may happen that the evaluation of amplification is not as clear as the examples discussed so far. Two such non-clear examples are shown in FIGS. 4a and 4b. In FIG. 4a, a value of 0.03 was obtained as first application-specific p-value p1 for the amplification present. This value is in the range of a possible limit value of 0.01 to 0.05 so that a further evaluation is useful depending on the defined limit value. The further evaluation resulted in a second application-specific p-value p2 with a value of 0.016, which indicates a significant amplification phase. A consideration of FIG. 4a shows that an amplification phase has actually occurred but only in later amplification cycles and with a small increase.

In FIG. 4b, a value of less than 0.01 was obtained as first application-specific p-value p1 for the amplification, which clearly indicates the presence of an amplification phase. However, the further evaluation resulted in a second application-specific p-value p2 with a value of 0.56, which contradicts the occurrence of an amplification phase. In this example, amplification was disrupted by an external error and the amplification phase is to be estimated as non-trustworthy as the second application-specific p-value p2 proves. In particular, in the case of a first application-specific p-value which is close to the limit value, it may be useful to ascertain the second application-specific p-value in order to make a statement on an occurrence of an amplification phase based on both p-values. As soon as one of the two application-specific p-values is significantly above the limit value, a lack of the amplification phase can then be assumed.

FIG. 5 shows in a graph how the starting point of the amplification phase is determined by means of a method of a sliding window. For this purpose, a defined number of measurement points MP ascertained in succession is taken from all measurement points MP and defined as a window. The window is divided into two halves and a specific parameter is determined in each of the first and second halves of the window. In this exemplary embodiment, in each of the two halves of the window comprising 14 measurement points MP, a line G1, G2 was placed through the seven measurement points MP in the respective half of the window, wherein the specific parameter corresponds to the increase in the line. Further possibilities for the specific parameter are an average value of the measurement points MP or a maximum value of the measurement points MP.

After determining the specific parameters in each of the two halves of the window, the window is shifted by a measurement point MP and the specific parameters are again determined in the two halves of the window. This step is repeated until all the specific parameters in all possible windows have been determined. The starting point is then that measurement point MP at which the difference in the specific parameters from the first and the second half of the window is greatest, i.e., for example, the increase in the lines G1, G2 in the first and the second half of the window has the greatest difference.

TABLE 1

| | p1 | p2 |
|---|---|---|
| FIG. 2a | <0.01 | <0.01 |
| FIG. 2b | <0.01 | <0.01 |
| FIG. 3a | 0.99 | — |
| FIG. 3b | 0.1 | — |
| FIG. 4a | 0.03 | 0.016 |
| FIG. 4b | <0.01 | 0.56 |

1. A method for detecting whether an amplification phase has occurred in an amplification in which at least one nucleic acid sequence is increased in a defined number of amplification cycles, an intensity of a signal of the at least one nucleic acid sequence being determined after each amplification cycle, the determined intensity represented as a measurement point as a function of the amplification cycle, the method comprising:
determining a starting point of the amplification phase based on the determined measurement points, the starting point marking a transition between a substantially constant background signal and a beginning of the amplification phase;
calculating a first regression line from a first measurement point recorded after a first amplification cycle to the determined starting point of the amplification phase;
calculating a second regression line over all measurement points;
comparing the first regression line to the second regression line with regard to dissimilarity between the first regression line and the second regression line using a first statistical test, wherein a first application-specific p-value is determined;
when the determined first application-specific p-value is above a defined first limit value:
detecting that no amplification phase has occurred; and
aborting a further evaluation; and
when the determined first application-specific p-value is below the first limit value:
detecting that an amplification phase has occurred and
performing a further evaluation.

2. The method of claim 1, the method further comprising:
adapting a first model function, which contains at least one sigmoid function, to all measurement points in a first step;
adapting a quadratic model function to all measurement points in a second step;
comparing the first model function to the quadratic model function with regard to dissimilarity between the first model function and the quadratic model function using a second statistical test, wherein a second application-specific p-value is determined;
when the determined second application-specific p-value is above a defined second limit value detecting that no amplification phase has occurred; and
when the determined second application-specific p-value is below the second limit value, detecting that an amplification phase has occurred.

3. The method of claim 1, wherein the first regression line and the second regression line are compared with regard to dissimilarity between the first regression line and the second regression line based on variances of residuals of the measurement points to the first regression line and to the second regression line using the first statistical test.

4. The method of claim 1, wherein the first statistical test is an F-test.

5. The method of claim 2, wherein the first model function and the quadratic model function are compared with regard to dissimilarity between the first model function and the quadratic model function based on variances of the residuals of the measurement points to the first model function and to the quadratic model function using the second statistical test.

6. The method of claim 2, wherein the second statistical test is an F-test.

7. The method of claim 2, wherein the first model function is formed as a sum of a sigmoid function and a linear function.

8. The method of claim 1, wherein a p-value of 0.05 or of 0.01, or between 0.01 and 0.05, is the first limit value of the determined first application-specific p-value.

9. The method of claim 1, wherein the first regression line is subtracted as background line from all the measurement points.

10. The method of claim 1, wherein the fluorescence or absorbance of the at least one nucleic acid sequence is measured as the signal intensity of the at least one nucleic acid sequence.

11. The method of claim 1, wherein a polymerase chain reaction is used as the amplification technique.

12. The method of claim 1, wherein the value of the determined first and/or second application-specific p-value is used to determine the quality of at least one quantification parameter, wherein the at least one quantification parameter is a measure for the concentration of the nucleic acid sequences and is determined based on the intensity of the signal of the at least one nucleic acid sequence.

13. The method of claim 12, wherein a threshold cycle and/or a quantification cycle is determined as at least one quantification parameter.

14. The method of claim 1, wherein the starting point of the amplification is determined using a method of a sliding window, wherein a defined number of measurement points determined in succession is taken from all measurement points to define a window, wherein a specific parameter is determined in each of a first half and a second half of the window, wherein the window is then shifted by a measurement point and a specific parameter is again determined in each of the first and second halves of the window, wherein the window is shifted by a measurement point in each case after the determination of the specific parameters until all such possible windows have been considered and all respective specific parameters have been determined, wherein the measurement point at which the difference between the specific parameters from the first and second halves of the window is greatest is selected as the starting point.

15. The method of claim 14, wherein an average value of the measurement points or a maximum value of the measurement points or an increase of a line formed between a first measurement point of the window and a middle measurement point of the window and between the middle measurement point of the window and a last measurement point of the window is calculated as a specific parameter.

16. The method of claim 2, wherein a p-value of 0.05 or of 0.01, or between 0.01 and 0.05, is the second limit value of the determined second application-specific p-value.
